(19) United States
(12) Patent Application Publication
(10) Pub. No.: US 2003/0190622 A1
(43) Pub. Date: Oct. 9, 2003

(54) Human kinase interacting protein 2 (KIP2)-related gene variant associated with lung cancers

(76) Inventor: Ken-Shwo Dai, Hsinchu (TW)

Correspondence Address: Ladas & Parry, 26 West 61st Street, New York, NY, 10023, US

(21) Appl. No.: 10/103,334

(22) Filed: Mar. 21, 2002

(51) Int. Cl. C12Q 1/68; G01N 33/574; C07H 21/04; C12P 21/02; C12N 5/06; C07K 14/47; C07K 16/18

(52) U.S. Cl. 435/6; 435/7.23; 435/69.1; 435/320.1; 435/325; 530/350; 530/388.1; 536/23.5

(57) ABSTRACT

The invention relates to the nucleic acid of a novel human KIP2-related gene variant (KIP2V) and the polypeptide encoded thereby.

The invention also provides a process for producing the polypeptide encoded by the KIP2V.

The invention further provides the uses of the nucleic acid of the KIP2V and the polypeptide encoded thereby in diagnosing diseases associated with the deficiency of human KIP2 gene, in particular lung cancers.

### FIELD OF THE INVENTION

The invention relates to the nucleic acid of a novel human kinase interacting protein 2 (KIP2)-related gene variant (KIP2V), the polypeptide encoded thereby, the preparation process thereof, and the uses of the same in diagnosing diseases associated with the deficiency of human KIP2 gene, in particular, lung cancers.

### BACKGROUND OF THE INVENTION

Lung cancer is one of the major causers of cancer-related deaths in the world. There are two primary types of lung cancers: small cell lung cancer (SCLC) and non-small cell lung cancer (NSCLC) (Carney, (1992a) Curr. Opin. Oncol. 4:292-8). Small cell lung cancer accounts for approximately 25% of lung cancer and spreads aggressively (Smyth et al. (1986) Q J Med. 61: 969-76; Carney, (1992b) Lancet 339: 843-6). Non-small cell lung cancer represents the majority (about 75%) of lung cancer and is further divided into three main subtypes: squamous cell carcinoma, adenocarcinoma, and large cell carcinoma (Ihde and Minna, (1991) Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy and radiotherapy. This is because the molecular mechanisms underlying the pathogenesis of lung cancers remain largely unclear.

A recent hypothesis suggests that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates, in particular, the genes localized on chromosome 11p15.5, a region shown to be associated with the development of lung cancer, in particular, the NSCLC (Bepler and Garcia-Blanco, (1994) Proc Natl Acad Sci USA 91:5513-7; Kondo et al. (1996) Oncogene 12:1365-8; Sanchez-Cespedes et al. (1997) Clin Cancer Res 3:1229-35; Pitterle et al. (1999) Mamm Genome 10:916-22). A human kinase interacting protein 2 (KIP2) gene, mapped on this region (Matsuoka et al. (1995) Genes Dev 9:650-62; Kondo et al. (1996) Oncogene 12:1365-8) is a member of Cip/Kip family involved in the negative regulation of the cell cycle at the G1 checkpoint (Lee et al. (1995) Genes Dev 9:639-49; Harper and Elledge, (1996) Curr Opin Genet Dev 6:56-64; Sherr, (1996) Science 274:1672-7; Lee and Yang, (2001) Cell Mol Life Sci 58:1907-22). The tumor suppressor activity of KIP2 was shown to be mediated via its interaction with cyclin A-CDK2 (Adkins and Lumb, (2002) Proteins 46:1-7) or proliferating cell nuclear antigen (PCNA; Watanabe et al. (1998) Proc Natl Acad Sci USA 95:1392-1397). Overexpression of KIP2 was reported to arrest cells in G1 (Lee et al. (1995) Genes Dev 9:639-49; Matsuoka et al. (1995) Genes Dev 9:650-62). Decreased expression of KIP2 has been observed in many cancers (Chung et al. (1996) Hum Mol Genet 5:1101-8; Oya and Schulz, (2000) Br J Cancer 83:626-31; Dauphinot et al. (2001) Oncogene 20:3258-65; Ito et al. (2001) Oncology 61:221-5). These findings strongly implicate that KIP2 may have a role in the tumorigenic process of NSCLC. Therefore, the discovery of gene variants of KIP2 from squamous cell lung cancer may be important targets for diagnostic markers of squamous cell lung cancer.

### SUMMARY OF THE INVENTION

The present invention provides a KIP2-related gene variant (KIP2V) present in human squamous cell lung cancer. The nucleotide sequence of KIP2V and the polypeptide sequence encoded thereby can be used for the diagnosis of diseases associated with the deficiency of human KIP gene, in particular, lung cancers, preferably the squamous cell lung cancer.

The invention further provides an expression vector and host cell for expressing KIP2V.

The invention further provides a method for producing the polypeptide encoded by KIP2V.

The invention further provides an antibody specifically binding to w the polypeptide encoded by KIP2V.

The invention also provides methods for diagnosing diseases associated with the deficiency of human KIP2 gene, in particular, lung cancers, preferably the squamous cell lung cancer.

### BRIEF DESCRIPTION OF THE DRAWINGS

FIGS. 1A and 1B show the nucleic acid sequence of KIP2V (SEQ ID NO:1) and the corresponding amino acid sequence thereof (SEQ ID NO:2).

FIGS. 2A to 2F show the nucleotide sequence alignment between the human KIP2 gene and KIP2V.

FIGS. 3A and 3B show the amino acid sequence alignment between the human KIP2 protein and the polypeptide encoded by KIP2V.

### DETAILED DESCRIPTION OF THE INVENTION

According to the present invention, all technical and scientific terms used have the same meanings as commonly understood by persons skilled in the art.

The term “antibody,” as used herein, denotes intact molecules (a polypeptide or group of polypeptides) as well as fragments thereof, such as Fab, R(ab′)₂, and Fv fragments, which are capable of binding the epitopic determinutesant. Antibodies are produced by specialized B cells after stimulation by an antigen. Structurally, antibody consists of four subunits including two heavy chains and two light chains. The internal surface shape and charge distribution of the antibody binding domain are complementary to the features of an antigen. Thus, antibody can specifically act against the antigen in an immune response.

The term “base pair (bp),” as used herein, denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402),” as used herein, denotes programs for evaluation of homologies between a query sequence (amino or nucleic acid) and a test sequence as described by Altschul et al. (Nucleic Acids Res. 25: 3389-3402, 1997). Specific BLAST programs are described as follows:

(1) BLASTN compares a nucleotide query sequence against a nucleotide sequence database;

(2) BLASTP compares an amino acid query sequence against a protein sequence database;

(3) BLASTX compares the six-frame conceptual translation products of a query nucleotide sequence against a protein sequence database;

(4) TBLASTN compares a query protein sequence against a nucleotide sequence database translated in all six reading frames; and

(5) TBLASTX compares the six-frame translations of a nucleotide query sequence against the six-frame translations of a nucleotide sequence database.

The term “cDNA,” as used herein, denotes nucleic acids that synthesized from a mRNA template using reverse transcriptase.

The term “cDNA library,” as used herein, denotes a library composed of complementary DNAs which are reverse-transcribed from mRNAs.

The term “complement,” as used herein, denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion,” as used herein, denotes a removal of a portion of one or more amino acid residues/nucleotides from a gene.

The term “expressed sequence tags (ESTs),” as used herein, denotes short (200 to 500 base pairs) nucleotide sequence that derives from either 5′ or 3′ end of a cDNA.

The term “expression vector,” as used herein, denotes nucleic acid constructs which contain a cloning site for introducing the DNA into vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell,” as used herein, denotes a cell which is used to receive, maintain, and allow the reproduction of an expression vector comprising DNA. Host cells are transformed or transfected with suitable vectors constructed using recombinant DNA methods. The recombinant DNA introduced with the vector is replicated whenever the cell divides.

The term “insertion” or “addition,” as used herein, denotes the addition of a portion of one or more amino acid residues/nucleotides to a gene.

The term “in silico,” as used herein, denotes a process of using computational methods (e.g., BLAST) to analyze DNA sequences.

The term “polymerase chain reaction (PCR),” as used herein, denotes a method which increases the copy number of a nucleic acid sequence using a DNA polymerase and a set of primers (about 20 bp oligonucleotides complementary to each strand of DNA) under suitable conditions (successive rounds of primer annealing, strand elongation, and dissociation).

The term “protein” or “polypeptide,” as used herein, denotes a sequence of amino acids in a specific order that can be encoded by a gene or by a recombinant DNA. It can also be chemically synthesized.

The term “nucleic acid sequence” or “polynucleotide,” as used herein, denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR),” as used herein, denotes a process which transcribes mRNA to complementary DNA strand using reverse transcriptase followed by polymerase chain reaction to amplify the specific fragment of DNA sequences.

The term “transformation,” as used herein, denotes a process describing the uptake, incorporation, and expression of exogenous DNA by prokaryotic host cells.

The term “transfection,” as used herein, a process describing the uptake, incorporation, and expression of exogenous DNA by eukaryotic host cells.

The term “variant,” as used herein, denotes a fragment of sequence (nucleotide or amino acid) inserted or deleted by one or more nucleotides/amino acids.

In the first aspect, the present invention provides the polypeptide encoded by a novel human KIP2-related gene variant (KIP2V) and the fragments thereof, as well as the nucleic acid sequence of KIP2V.

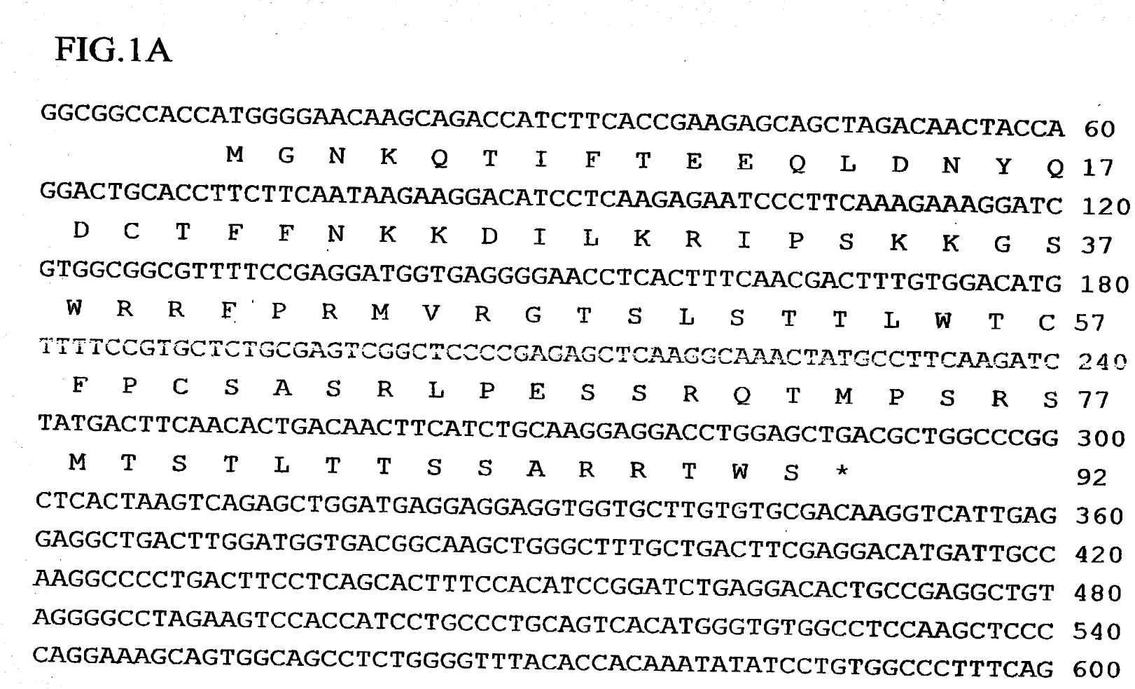

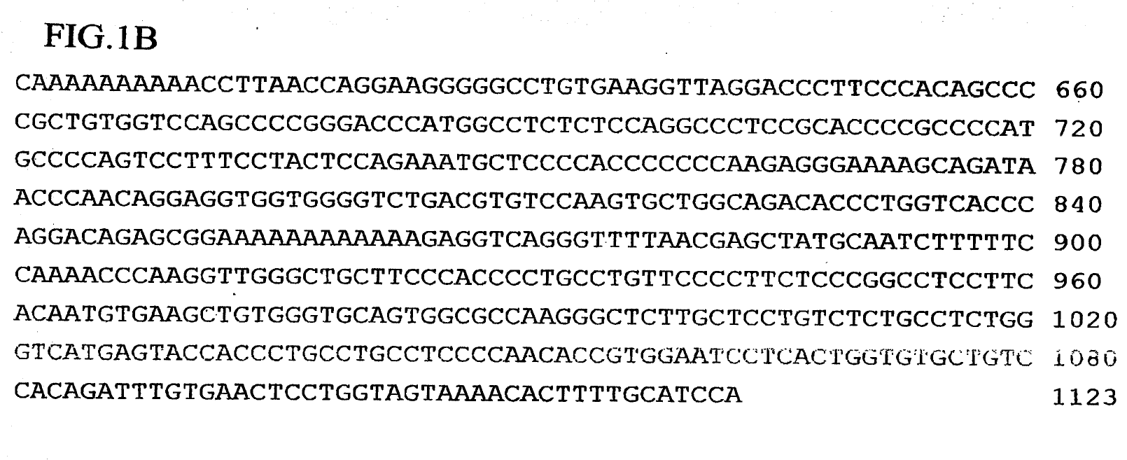

According to the present invention, human KIP2 cDNA sequence was used to query the human lung EST databases (a normal lung, a large cell lung cancer, a squamous cell lung cancer and a small cell lung cancer) using BLAST program to search for KIP2-related gene variants. One human cDNA partial sequences (i.e., EST) showing similarity to KIP2 was identified from ESTs deposited in the squamous cell lung cancer database. The cDNA clone, named KIP2V (KIP2 gene variant), was then isolated from the the squamous cell lung cancer cDNA library and sequenced. FIGS. 1A and 1B show the nucleic acid sequence (SEQ ID NO:1) of KIP2V and its corresponding amino acid sequence (SEQ ID NO:2).

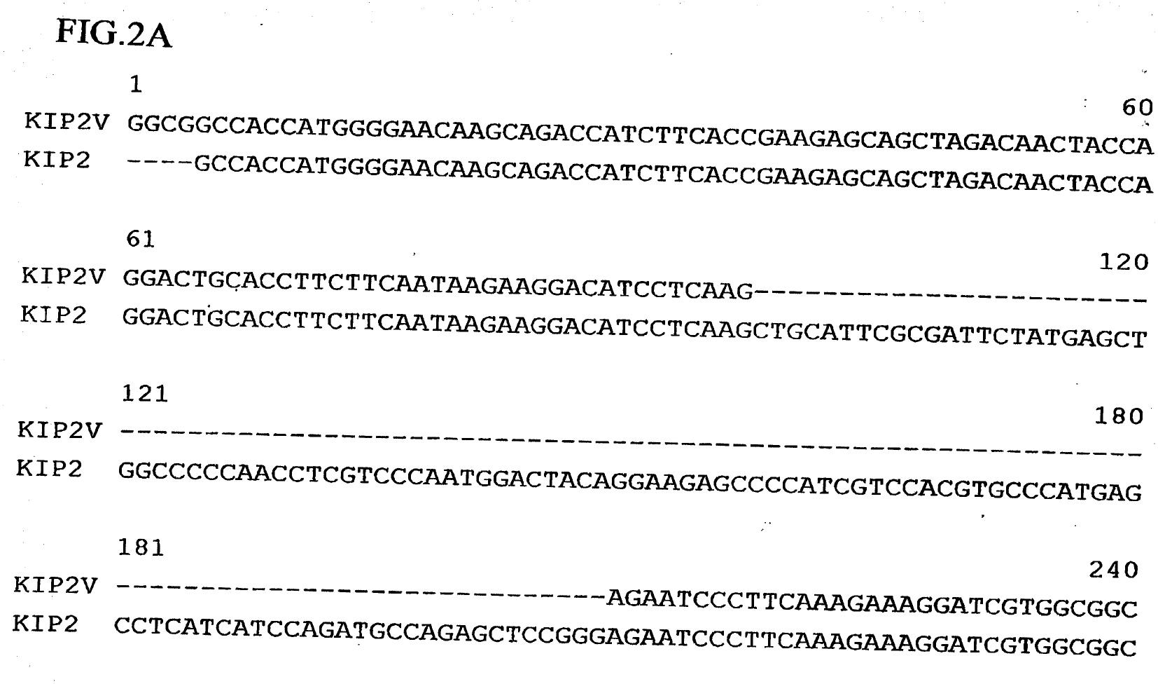

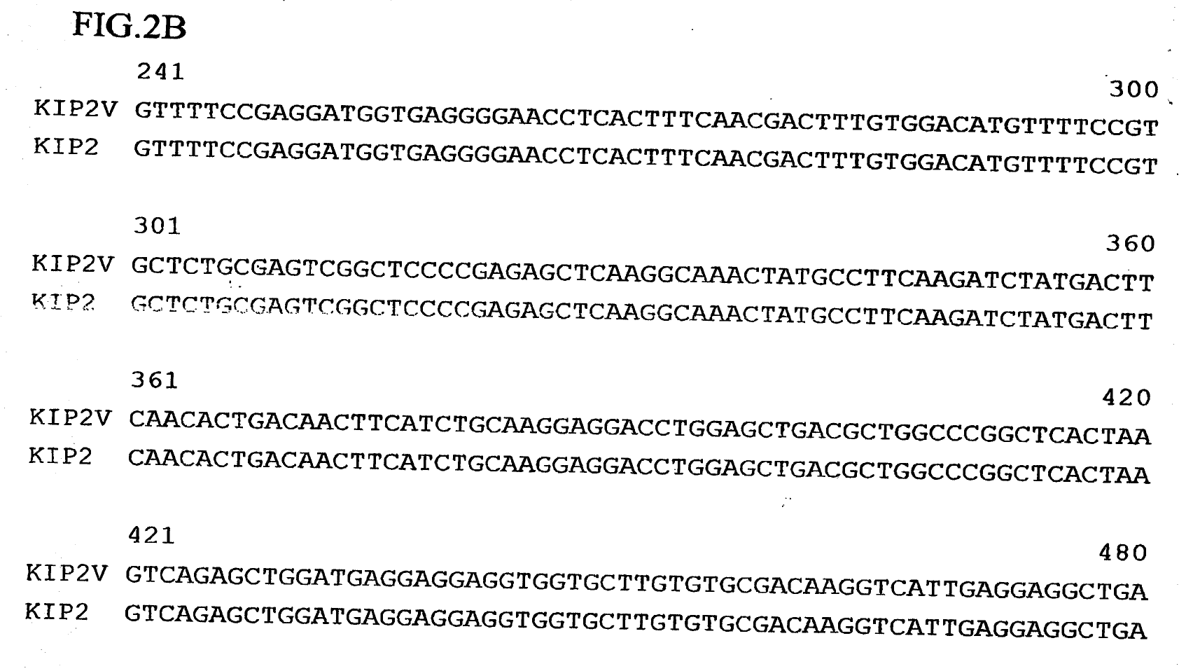

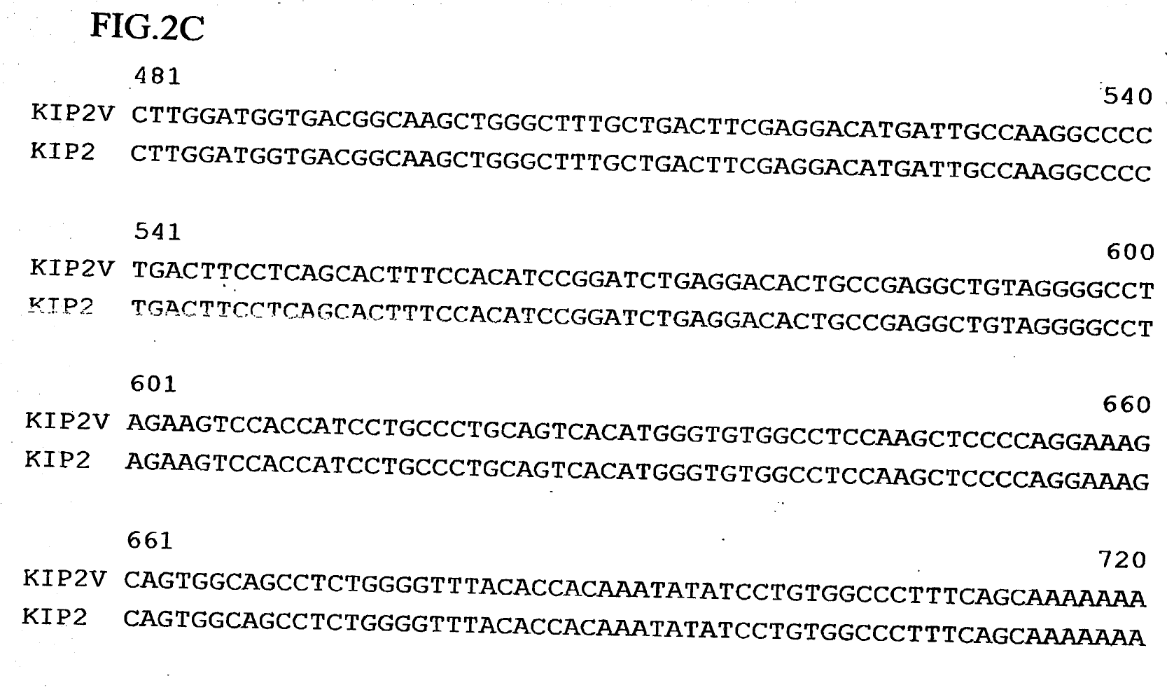

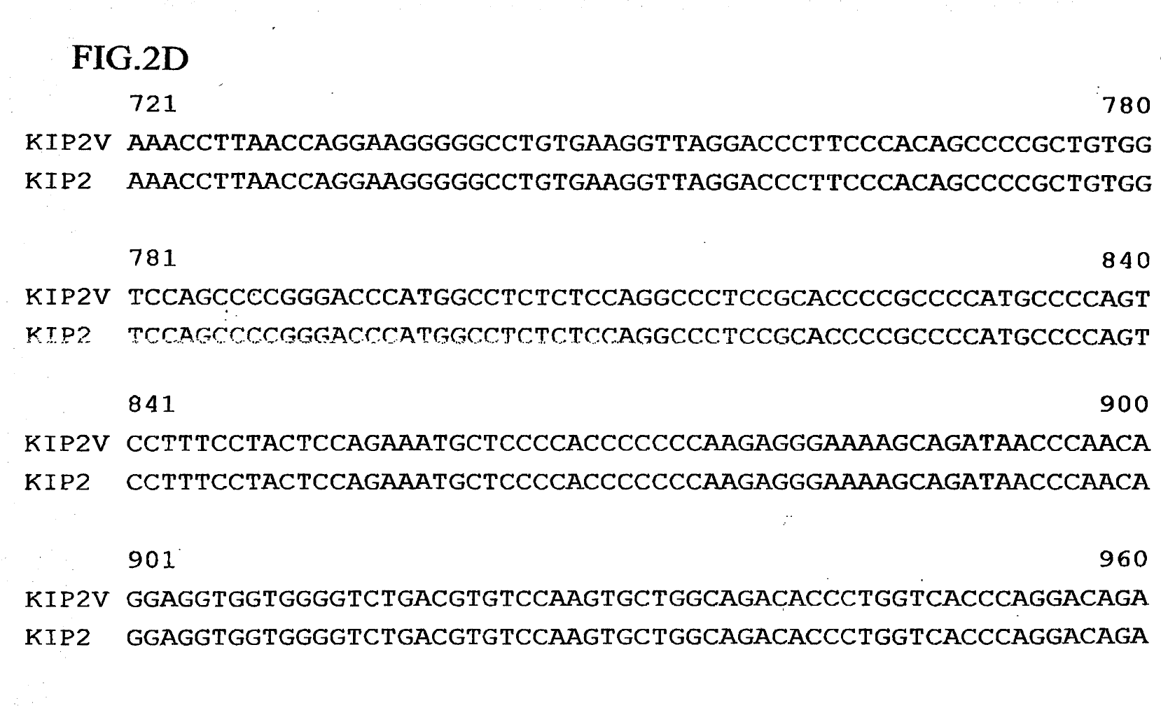

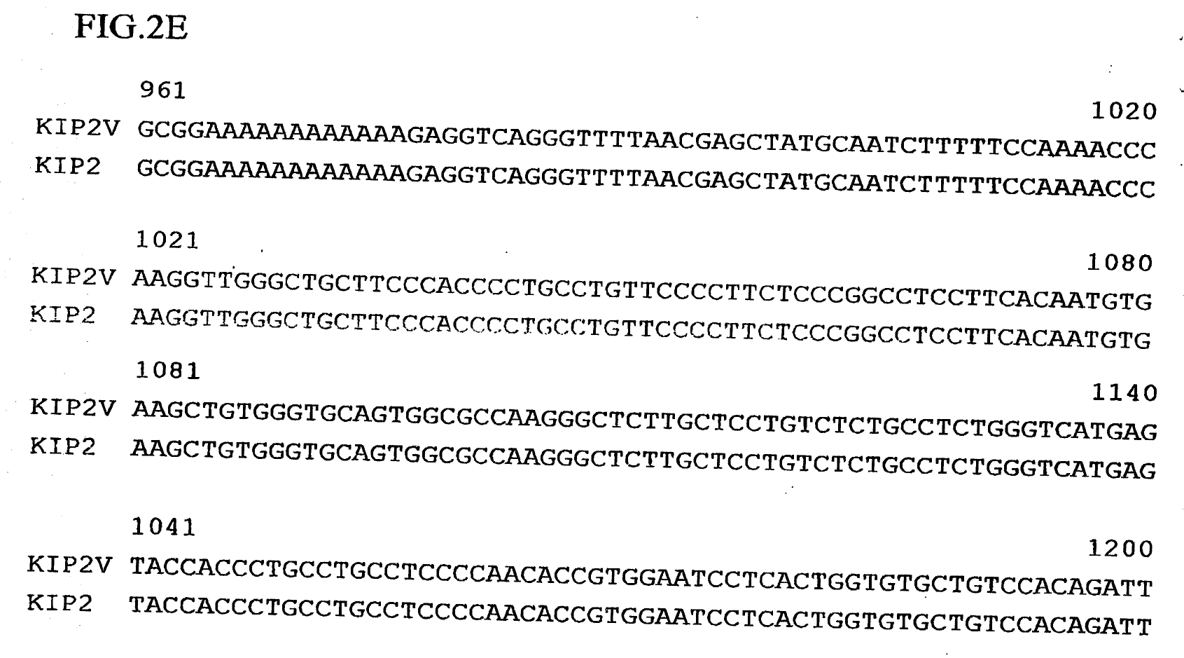

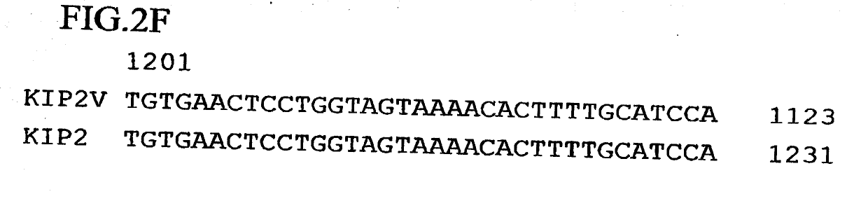

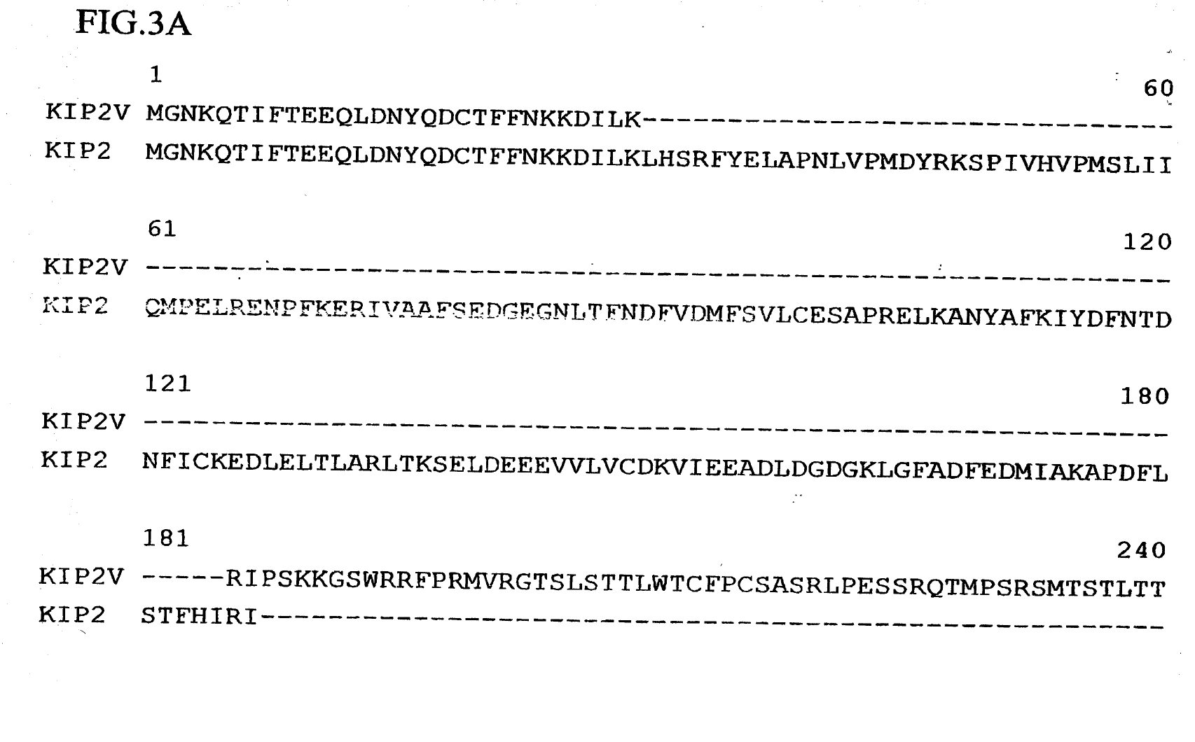

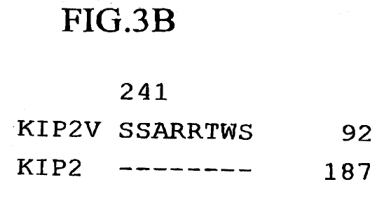

The full-length of the KIP2V cDNA is an 1123 bp clone containing a 276 bp open reading frame (ORF) extending from 11 bp to 286 bp, which corresponds to an encoded protein of 92 amino acid residues with a predicted molecular mass of 10.7 kDa. The sequence around the initiation ATG codon of KIP2V (located at nucleotide 11 to 13 bp) was matched to the Kozak consensus sequence (A/GCCATGG) (Kozak, (1987) Nucleic Acids Res. 15: 8125-48; Kozak, (1991) J Cell Biol. 115: 887-903.). To determine the variation in sequence of KIP2V cDNA clone, an alignment of KIP2 nucleotide/amino acid sequence with KIP2V was performed (FIGS. 2 and 3). One major genetic deletion was found in the aligned sequences, which shows that KIP2V is an 112 bp deletion in the sequence of KIP2 from nucleotides 94 to 205. The lacking of 112 bp generates a frame-shift starting from the amino acid position 30 of KIP2. The predicted amino acid sequence indicates that only the N-terminal section (the first 29 amino acids) of KIP2 is still conserved on KIP2V protein.

In the present invention, a search of ESTs deposited in dbEST (Boguski et al. (1993) Nat Genet. 4: 332-3) at National Center for Biotechnology Information (NCBI) was performed to determine the tissue distribution of V in silico. The result of in silico Northern analysis showed that one EST (GenBank accession number AI817087) was found to confirm the absence of 112 bp region on KIP2V nucleotide sequence. This EST was generated from a squamous cell lung cancer cDNA library suggesting that the absence of 112 bp nucleotide fragment located between nucleotides 97 to 98 of KIP2V may serve as a useful marker for diagnosing squamous cell lung cancer. Therefore, any nucleotide fragments comprising nucleotides 97 to 98 of KIP2V may be used as probes for determining the presence of KIP2V under high stringency conditions. An alternative approach is that any set of primers for amplifying the fragment containing nucleotides 97 to 98 of KIP2V may be used for determining the presence of the variant.

Scanning the KIP2V sequence against the profile entries in PROSITE (ScanProsite) indicated that KIP2V protein contains four protein kinase C phosphorylation sites (33-35aa, 37-39aa, 68-70aa and 86-88aa), one cAMP- and cGMP-dependent protein kinase phosphorylation site (34-37aa), two N-myristoylation sites (2-7aa, and 47-52aa), and one Signal peptidases I serine active site (75-82aa).

According to the present invention, the polypeptide encoded by KIP2V and its fragments thereof may be produced through genetic engineering techniques. For instance, they may be produced by using appropriate host cells which have been transformed with recombinant DNAs that code for the desired polypeptides or fragments thereof. The nucleotide sequence of KIP2V or the fragments thereof is inserted into an appropriate expression vector, i.e., a vector which contains the necessary elements for the transcription and translation of the inserted coding sequence in a suitable host. The nucleotide sequence is inserted into the vector in a manner that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing the sequence of KIP2V and appropriate transcriptional/translational control elements. These methods may include in vitro recombinant DNA and synthetic techniques, and in vivo genetic recombinant techniques. (See, e.g., Sambrook, J. Cold Spring Harbor Press, Plainview N.Y., Ch. 4, 8, and 16-17; Ausubel, R. M. et al. (1995) Current protocols in Molecular Biology, John Wiley & Sons, New York N.Y., ch. 9, 13, and 16.)

A variety of expression vector/host systems may be utilized to express KIP2V. These include, but not limited to, microorganisms such as bacteria transformed with recombinant bacteriophage, plasmid, or cosmid DNA expression vector; yeast transformed with yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with virus (e.g., vaccina virus, adenovirus, etc.). Preferably, the host cell is a bacterium, and more preferably, the bacterium is E. coli.

Alternatively, the polypeptide encoded by KIP2V or fragments thereof may be synthesized using chemical methods. For example, peptide synthesis can be performed using various solid-phase techniques (Roberge, J. Y. et al. (1995) Science 269: 202 to 204). Automated synthesis may be achieved using the ABI 431A peptide synthesizer (Perkin-Elmer).

According to the present invention, the fragments of the nucleotide sequence of KIP2V and the polypeptide encoded thereby are used as primers or probes and immunogens, respectively. Preferably, the purified fragments are used. The fragments may be produced by enzyme digestion, chemical cleavage of isolated or purified polypeptide or nucleotide sequences, or chemical synthesis and then may be isolated or purified. Such isolated or purified fragments of the polypeptides and nucleotide sequences can be used directed as immunogens and primers or probes, respectively.

The present invention further provides the antibodies which specifically bind to one or more out-surface epitopes of the polypeptide encoded by KIP2V.

According to the present invention, immunization of mammals with immunogens described herin, preferably humans, rabbits, rats, mice, sheep, goats, cows, or horses, is performed by following the procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. Nos. 4,271,145 and 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridoma clones are isolated, and the monoclonal antibodies are recovered from those clones.

Immunization regimens for production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the present invention, antibody fragments which contain specific binding sites for the polypeptides or fragments thereof may also be generated. For example, such fragments include, but are not limited to, F(ab′)₂ fragments produced by pepsin digestion of the antibody molecule and Fab fragments generated by reducing the disulfide bridges of the F(ab′)₂ fragments.

Many gene variants have been found to be associated with diseases (Stallings-Mann et al., (1996) Proc Natl Acad Sci USA 93: 12394-9; Liu et al., (1997) Nat Genet 16:328-9; Siffert et al., (1998) Nat Genet 18: 45 to 8; Lukas et al., (2001) Cancer Res 61: 3212 to 9). Since KIP2V clone was isolated from squamous cell lung cancer cDNA library and its expression in squamous cell lung cancer was confirmed by in silico Northern analysis, it is advisable that KIP2V may serve as markers for the diagnosis of diseases associated with the deficiency of human KIP2 gene, in particular lung cancers, e.g. human squamous cell lung cancer. Thus, the expression level of KIP2V relative to KIP2 may be a useful indicator for screening of patients suspected of having such diseases, and the index of relative expression level (mRNA or protein) may confer an increased susceptibility to the same.

Accordingly, the subject invention further provides methods for diagnosing the diseases associated with the deficiency of human KIP2 gene in a mammal, in particular, lung cancers, preferably the squamous cell lung cancer.

The method for diagnosing the diseases associated with the deficiency of human KIP2 gene may be performed by detecting the nucleotide sequence of the KIP2V of the invention, which comprises the steps of: (1) extracting the total RNA of cells obtained from the mammal; (2) amplifying the RNA by reverse transcriptase-polymerase chain reaction (RT-PCR) with a set of primers to obtain a cDNA comprising the fragments comprising nucleotides 95 to 100 of SEQ ID NO: 1; and (3) detecting whether the cDNA is obtained. If necessary, the amount of the obtained cDNA sample may be determined.

In this embodiment, one of the primers may be designed to have a sequence comprising the nucleotides of SEQ ID NO: 1 containing nucleotides 95 to 100, and the other may be designed to have a sequence complementary to the nucleotides of SEQ ID NO: 1 at any other locations downstream of nucleotide 100. Alternatively, one of the primers may be designed to have a sequence complementary to the nucleotides of SEQ ID NO: 1 containing nucleotides 95 to 100, and the other may be designed to have a sequence comprising the nucleotides of SEQ ID NO: 1 at any other locations upstream of nucleotide 95. In this case, only KIP2V will be amplified.

Alternatively, one of the primers may be designed to have a sequence comprising the nucleotides of SEQ ID NO: 1 upstream of nucleotide 97 and the other may be designed to have a sequence complementary to the nucleotides of SEQ ID NO: 1 downstream of nucleotide 98. Alternatively, one of the primers may be designed to have a sequence complementary to the nucleotides of SEQ ID NO: 1 upstream of nucleotide 97 and the other may be designed to have a sequence comprising the nucleotides of SEQ ID NO: 1 down stream of nucleotide 98. In this case, both KIP2 and KIP2V will be amplified. The length of the PCR fragment from KIP2V will be 112 bp shorter than that from KIP2.

Preferably, the primer of the invention contains 15 to 30 nucleotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected sizes of specific PCR fragments of KIP2V can be used. PCR fragments are analyzed on a 1% agarose gel using five microliters (10%) of the amplified products. To determine the expression levels for each gene variants, the intensity of the PCR products may be determined by using the Molecular Analyst program (version 1.4.1; Bio-Rad).

The RT-PCR experiment may be performed according to the manufacturer instructions (Boehringer Mannheim). A 50 μl reaction mixture containing 2 μl total RNA (0.1 μg/μl), 1 μl each primer (20 pM), 1 μl each dNTP (10 mM), 2.5 μl DTT solution (100 mM), 10 μl 5×RT-PCR buffer, 1 μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment of the method for diagnosing the diseases associated with the deficiency of human KIP2 gene may be performed by detecting the nucleotide sequence of KIP2V, which comprises the steps of: (1) extracting total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid of SEQ ID NO: 1 or the fragments thereof; and (4) detecting whether the cDNA sample hybridizes with the nucleic acid. If necessary, the amount of the hybridized sample may be determined.

The expression of gene variants can also be analyzed using Northern blot hybridization approach. Specific fragments comprising nucleotides 95 to 100 of KIP2V may be amplified by polymerase chain reaction (PCR) using primer set designed for RT-PCR. The amplified PCR fragment may be labeled and serve as a probe to hybridize the membranes containing total RNAs extracted from the samples under the conditions of 55° C. in a suitable hybridization solution for 3 hr. Blots may be washed twice in 2×SSC, 0.1% SDS at room temperature for 15 minutes each, followed by two washes in 0.1×SSC and 0.1% SDS at 65° C. for 20 minutes each. After these washes, blot may be rinsed briefly in suitable washing buffer and incubated in blocking solution for 30 minutes, and then incubated in suitable antibody solution for 30 minutes. Blots may be washed in washing buffer for 30 minutes and equilibrated in suitable detection buffer before detecting the signals. Alternatively, the presence of gene variants (cDNAs or PCR) can be detected using microarray approach. The cDNAs or PCR products corresponding to the nucleotide sequences of the invention may be immobilized on a suitable substrate such as a glass slide. Hybridization can be preformed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the invention, the method for diagnosing the diseases associated with the deficiency of human KIP2 gene may also be performed by detecting the polypeptide encoded by the KIP2V of the invention. For instance, the polypeptide in protein samples obtained from the mammal may be determined by, but not limited to, the immunoassay wherein the antibodies specifically binding to the polypeptides of the invention is contacted with the sample, and the antibody-polypeptide complex is detected. If necessary, the amount of antibody-polypeptide complex can be determined.

The polypeptide encoded by KIP2V may be expressed in prokaryotic cells by using suitable prokaryotic expression vectors. The cDNA fragments of KIP2V may be PCR-amplified using primer set with restriction enzyme digestion sites incorporated in the 5′ and 3′ ends, respectively. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3 h. The bacterially-expressed proteins may be purified.

The polypeptide encoded by the KIP2V may be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% CO₂ atmosphere. Before transfection, the nucleotide sequence of each of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated in 12-well plates one day before transfection at a density of 5×10⁴ cells per well. Transfections may be carried out using Lipofectaminutese Plus transfection reagent according to the manufacturer's instructions (Gibco BRL). Three hours following transfection, medium containing the complexes may be replaced with fresh medium. Forty-eight hours after incubation, the cells may be scraped into lysis buffer (0.1 M Tris HCl, pH 8.0, 0.1% Triton X-100) for purification of expressed proteins. After these proteins are purified, monoclonal antibodies against these purified proteins may be generated using hybridoma technique according to the conventional methods (de StGroth and Scheidegger, (1980) J Immunol Methods 35:1-21; Cote et al. (1983) Proc Natl Acad Sci USA 80: 2026-30; and Kozbor et al. (1985) J Immunol Methods 81:31-42).

According to the invention, the presence of the polypeptide encoded by KIP2V in samples of normal lung and lung cancers may be determined by, but not limited to, Western blot analysis. Proteins extracted from samples may be separated by SDS-PAGE and transferred to suitable membranes such as polyvinylidene difluoride (PVDF) in transfer buffer (25 mM Tris-HCl, pH 8.3, 192 mM glycine, 20% methanol) with a Trans-Blot apparatus for 1 h at 100 V (e.g., Bio-Rad). The proteins can be immunoblotted with specific antibodies. For example, membrane blotted with extracted proteins may be blocked with suitable buffers such as 3% solution of BSA or 3% solution of nonfat milk powder in TBST buffer (10 mM Tris-HCl, pH 8.0, 150 mM NaCl, 0.1% Tween 20) and incubated with monoclonal antibody specific to the polypeptide encoded by these gene variants. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

### Analysis of Human Lung EST Databases

Expressed sequence tags (ESTs) generated from the large-scale PCR-based sequencing of the 5′-end of human lung (normal, SCLC, squamous cell lung cancer and large cell lung cancer) cDNA clones were compiled and served as EST databases. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the National Center for Biotechnology Information (NCBI) with a significance cutoff of p<10⁻¹⁰. ESTs representing putative KIP2V gene were identified during the course of EST generation.

### Isolation of cDNA Clones

One cDNA clone exhibiting EST sequence similar to the KIP2 gene was isolated from the squamous cell lung cancer cDNA library and named KIP2V. The inserts of these clones were subsequently excised in vivo from the λZAP Express vector using the ExAssist/XLOLR helper phage system (Stratagene). Phagemid particles were excised by coinfecting XL1-BLUE MRF′ cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Epicentre#SE9101LC SequiTherm EXCEL™II DNA Sequencing Kit for 4200S-2 Global NEW IR² DNA sequencing system (LI-COR). Using the primer-walking approach, fill-length sequence was determined. Nucleotide and protein searches were performed using BLAST against the non-redundant database of NCBI.

### In Silico Tissue Distribution (Northern) Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variants (insertions or deletions) with a significance cutoff of p<10⁻¹⁰.

### REFERENCES

Adkins and Lumb, Intrinsic structural disorder and sequence features of the cell cycle inhibitor p57(Kip2). Proteins, 46:1-7 (2002).

Altschul et al., Gapped BLAST and PSI-BLAST: a new generation of protein database search programs, Nucleic Acids Res, 25: 3389-3402, (1997).

Ausubel et al., Current protocols in Molecular Biology, John Wiley & Sons, New York N.Y., ch. 9, 13, and 16, (1995).

Bepler and Garcia-Blanco, Three tumor-suppressor regions on chromosome 11 p identified by high-resolution deletion mapping in human non-small-cell lung cancer. Proc Natl Acad Sci USA, 91:5513-7 (1994).

Boguski et al., dbEST—database for “expressed sequence tags”. Nat Genet. 4: 332-3, (1993).

Carney, D. N. The biology of lung cancer. Curr. Opin. Oncol. 4: 292-8, (1992a).

Carney, D. N. Biology of small-cell lung cancer. Lancet 339: 843-6, (1992b).

Chung et al. Chromosome 11p15.5 regional imprinting: comparative analysis of KIP2 and H19 in human tissues and Wilms' tumors. Hum Mol Genet 5:1101-8 (1996).

Cote et al., Generation of human monoclonal antibodies reactive with cellular antigens, Proc Natl Acad Sci USA 80: 2026-30 (1983).

Dauphinot et al. Analysis of the expression of cell cycle regulators in Ewing cell lines: EWS-FLI-1 modulates p57KIP2 and c-Myc expression. Oncogene 20:3258-65, (2001)

de StGroth and Scheidegger, Production of monoclonal antibodies: strategy and tactics, J Immunol Methods 35:1-21, (1980).

Gish and States, Identification of protein coding regions by database similarity search, Nat Genet, 3:266-272, (1993).

Harper and Elledge, Cdk inhibitors in development and cancer. Curr Opin Genet Dev 6:56-64, (1996).

Ihde and Minna, Non-small cell lung cancer. Part II: Treatment. Curr. Probl. Cancer 15: 105-54, (1991).

Ito et al., Expression of p57/Kip2 protein in hepatocellular carcinoma. Oncology, 61:221-5, (2001)

Kondo et al., Selective maternal-allele loss in human lung cancers of the maternally expressed p57KIP2 gene at 11p15.5. Oncogene 12:1365-8, (1996).

Kozak, An analysis of 5′-noncoding sequences from 699 vertebrate messenger RNAs. Nucleic Acids Res, 15: 8125-48, (1987).

Kozak, An analysis of vertebrate mRNA sequences: intimations of translational control, J Cell Biol, 115: 887-903, (1991).

Kozbor et al., Specific immunoglobulin production and enhanced tumorigenicity following ascites growth of human hybridomas, J Immunol Methods, 81:31-42 (1985).

Lee and Yang, Negative regulators of cyclin-dependent kinases and their roles in cancers. Cell Mol Life Sci 58:1907-22, (2001).

Lee et al., Cloning of p57KIP2, a cyclin-dependent kinase inhibitor with unique domain structure and tissue distribution. Genes Dev 9:639-49, (1995).

Liu et al., Silent mutation induces exon skipping of fibrillin-1 gene in Marfan syndrome. Nat Genet 16:328-9, (1997).

Lukas et al., Alternative and aberrant messenger RNA splicing of the mdm2 oncogene in invasive breast cancer. Cancer Res 61:3212-9, (2001).

Matsuoka et al. p57KIP2, a structurally distinct member of the p21CIP1 Cdk inhibitor family, is a candidate tumor suppressor gene. Genes Dev 9:650-62, (1995).

Oya and Schulz, Decreased expression of p57(KIP2) mRNA in human bladder cancer. Br J Cancer 83:626-31, (2000).

Pitterle et al. Lung cancer and the human gene for ribonucleotide reductase subunit M1 (RRM1). Mamm Genome 10:916-22, (1999).

Roberge et al., A strategy for a convergent synthesis of N-linked glycopeptides on a solid support. Science 269:202-4, (1995).

Sambrook, J. Cold Spring Harbor Press, Plainview N.Y., ch. 4, 8, and 16-17.

Sanchez-Cespedes et al. Microsatellite alterations at 5q21, 11p13, and 11p15.5 do not predict survival in non-small cell lung cancer. Clin Cancer Res 3:1229-35, (1997).

Sethi, Science, medicine, and the future. Lung cancer, BMJ, 314: 652-655, (1997)

Sherr, Cancer cell cycles. Science 274:1672-7, (1996).

Siffert et al., Association of a human G-protein beta3 subunit variant with hypertension. Nat Genet, 18:45-8, (1998).

Smyth et al., The impact of chemotherapy on small cell carcinoma of the bronchus. Q J Med, 61: 969-76, (1986).

Stallings-Mann et al., Alternative splicing of exon 3 of the human growth hormone receptor is the result of an unusual genetic polymorphism. Proc Natl Acad Sci USA 93:12394-9, (1996).

Strausberg, R. EST Accession No. AI817087

Watanabe et al. Suppression of cell transformation by the cyclin-dependent kinase inhibitor p57KIP2 requires binding to proliferating cell nuclear antigen. Proc Natl Acad Sci USA, 95:1392-1397, (1998).

What is claimed is:

1. An isolated polypeptide comprising the amino acid sequence of SEQ ID NO: 2, and fragments thereof.

2. An isolated nucleic acid encoding the polypeptide of claim 1, and fragments thereof.

3. The isolated nucleic acid of claim 2, which comprises the nucleotide sequence of SEQ ID NO: 1.

4. The isolated nucleic acid of claim 3, wherein the fragments comprise the nucleotides 95 to 100 of SEQ ID NO: 1.

5. An expression vector comprising the nucleic acid of any one of claims 2 to 4.

6. A host cell transformed with the expression vector of claim 5.

7. A method for producing the polypeptide of claim 1, which comprises the steps of:
(1) culturing the host cell of claim 6 under a condition suitable for the expression of the polypeptide; and
(2) recovering the polypeptide from the host cell culture.

8. An antibody specifically binding to the polypeptide of claim 1.

9. A method for diagnosing the diseases associated with the deficiency of KIP2V gene in a mammal, in particular lung cancers, which comprises detecting the nucleic acid of any one of claims 2 to 4 or the polypeptide of claim 1.

10. The method of claim 9, wherein the lung cancer is squamous cell lung cancer.

11. The method of claim 9, wherein the detection of the nucleic acid of any one claims 2 to 4 comprises the steps of:
(1) extracting total RNA from a sample obtained from the mammal;
(2) amplifying the RNA by reverse transcriptase-polymerase chain reaction (RT-PCR) with a pair of primers to obtain a cDNA sample comprising the nucleotides 95 to 100 of SEQ ID NO: 1; and
(3) detecting whether the cDNA sample is obtained.

12. The method of claim 11, wherein one of the primers has a sequence comprising the nucleotides of SEQ ID NO: 1 containing nucleotides 95 to 100, and the other has a sequence complementary to the nucleotides of SEQ ID NO: 1 at any other locations downstream of nucleotide 100, or one of the primers has a sequence complementary to the nucleotides of SEQ ID NO: 1 containing nucleotides 95 to 100, and the other has a sequence comprising the nucleotides of SEQ ID NO: 1 at any other locations up stream of nucleotide 95.

13. The method of claim 11, wherein one of the primers has a sequence comprising the nucleotides of SEQ ID NO: 1 upstream of nucleotide 97 and the other has a sequence complementary to the nucleotides of SEQ ID NO: 1 downstream of nucleotide 98, or one of the primers has a sequence complementary to the nucleotides of SEQ ID NO: 1 upstream of nucleotide 97 and the other has a sequence comprising the nucleotides of SEQ ID NO: 1 downstream of nucleotide 98.

14. The method of claim 13, wherein the cDNA sample amplified from SEQ ID NO: 1 is 112 bp shorter than the cDNA sample amplified from human KIP2 gene.

15. The method of claim 11 further comprising the step of detecting the amount of the amplified cDNA sample.

16. The method of claim 9, wherein the detection of the nucleic acid of any one of claims 2 to 4 comprises the steps of:
(1) extracting the total RNA of a sample obtained from the mammal;
(2) amplifying the RNA by reverse transcriptase-polymerase chain reaction (RT-PCR) to obtain a cDNA sample;
(3) bringing the cDNA sample into contact with the nucleic acid of any one of claims 2 to 4; and
(4) detecting whether the cDNA sample hybridizes with the nucleic acid of any one of claims 2 to 4.

17. The method of claim 16 further comprising the step of detecting the amount of hybridized sample.

18. The method of claim 9, wherein the detection of the polypeptide of claim 1 comprises the steps of contacting the antibody of claim 8 with a protein sample obtained from the mammal, and detecting whether an antibody-polypeptide complex is formed.

19. The method of claim 18 further comprising the step of detecting the amount of the antibody-polypeptide complex.


SEQUENCE LISTING (2 sequences)

SEQ ID NO: 1 - DNA, 1123 bp, Homo sapiens
ggcggccaccatggggaacaagcagaccatcttcaccgaagagcagctagacaactaccaggactgcaccttcttcaataagaaggacatcctcaagagaatcccttcaaagaaaggatcgtggcggcgttttccgaggatggtgaggggaacctcactttcaacgactttgtggacatgttttccgtgctctgcgagtcggctccccgagagctcaaggcaaactatgccttcaagatctatgacttcaacactgacaacttcatctgcaaggaggacctggagctgacgctggcccggctcactaagtcagagctggatgaggaggaggtggtgcttgtgtgcgacaaggtcattgaggaggctgacttggatggtgacggcaagctgggctttgctgacttcgaggacatgattgccaaggcccctgacttcctcagcactttccacatccggatctgaggacactgccgaggctgtaggggcctagaagtccaccatcctgccctgcagtcacatgggtgtggcctccaagctccccaggaaagcagtggcagcctctggggtttacaccacaaatatatcctgtggccctttcagcaaaaaaaaaaccttaaccaggaagggggcctgtgaaggttaggacccttcccacagccccgctgtggtccagccccgggacccatggcctctctccaggccctccgcaccccgccccatgccccagtcctttcctactccagaaatgctccccacccccccaagagggaaaagcagataacccaacaggaggtggtggggtctgacgtgtccaagtgctggcagacaccctggtcacccaggacagagcggaaaaaaaaaaaagaggtcagggttttaacgagctatgcaatctttttccaaaacccaaggttgggctgcttcccacccctgcctgttccccttctcccggcctccttcacaatgtgaagctgtgggtgcagtggcgccaagggctcttgctcctgtctctgcctctgggtcatgagtaccaccctgcctgcctccccaacaccgtggaatcctcactggtgtgctgtccacagatttgtgaactcctggtagtaaaacacttttgcatcca

SEQ ID NO: 2 - protein, 92 aa, Homo sapiens
MGNKQTIFTEEQLDNYQDCTFFNKKDILKRIPSKKGSWRRFPRMVRGTSLSTTLWTCFPCSASRLPESSRQTMPSRSMTSTLTTSSARRTWS